(19) United States
(12) Patent Application Publication
(10) Pub. No.: US 2010/0178274 A1
(43) Pub. Date: Jul. 15, 2010

(54) APPLICATION OF SYNOVIUM-DERIVED MESENCHYMAL STEM CELLS (MSCS) FOR CARTILAGE OR MENISCUS REGENERATION

(76) Inventors: Ichiro Sekiya, Tokyo (JP); Takeshi Muneta, Tokyo (JP); Tomohiro Morio, Tokyo (JP); Norio Shimizu, Yamanashi (JP); Yasuyuki Kuroiwa, Ibaraki (JP)

Correspondence Address: BIRCH STEWART KOLASCH & BIRCH, PO BOX 747, FALLS CHURCH, VA, 22040-0747, US

(21) Appl. No.: 12/438,298

(22) PCT Filed: Aug. 22, 2007
(86) PCT No.: PCT/JP2007/066708

§ 371 Date: Feb. 20, 2009

(30) Foreign Application Priority Data
Aug. 22, 2006 (US) 60823138

(51) Int. Cl. A61K 35/12; A61P 19/00

(52) U.S. Cl. 424/93.7

(57) ABSTRACT

An object of the present invention is to provide a method for treating defects of articular cartilage or meniscus of a patient using in vivo chondrogenesis of synovium-derived MSCs. The present invention provides a method for treating a disease associated with defects of cartilage or meniscus. In the present invention, the method for treating a disease associated with defects of cartilage or meniscus comprises the following steps: culturing ex vivo autologous synovium-derived mesenchymal stem cells (MSCs); implanting the MSCs such that said cartilage defect site or meniscal defect site is covered by the MSCs; and regenerating cartilage tissue at the cartilage defect site or meniscal defect site in situ by differentiating the MSCs into cartilage cells.

### BACKGROUND ART

This invention relates to a method for treating defects of the articular cartilage or meniscus of a patient using in vivo chondrogenesis of synovium-derived MSCs.

Articular cartilage defects and meniscal defects are associated with articular pain, decrease in range of motion, hydrarthrosis, mobility impairment and so on. A patient suffering from articular cartilage defects or meniscal defects caused by trauma is usually treated by an orthopaedic surgeon. Surgical repair of cartilage defects or meniscal defects aims to eliminate any loose debris that could cause further damage to the joint, and to restore function to the affected joint.

Some procedures are often recommended by orthopaedic surgeons for articular cartilage injury, depending on the severity of damage. Examples of procedures employed by orthopaedic surgeons include marrow stimulation, mosaicplasty (also referred to as osteochondral autograft, or bone/cartilage plug implantation), autologous chondrocyte implantation, and so on.

Marrow stimulation is a procedure which is used to promote cartilage repair by recruiting marrow-derived stem cells to an injury site. This procedure is conducted by puncturing or removing part of the subchondral bone plate to induce bleeding from the marrow cavity, and can be used to treat injury sites with a surface area of up to 2 cm². This procedure is advantageous in that it is simple and can be performed arthroscopically, but is disadvantageous in that defects are repaired by fibrous cartilage rather than hyaline cartilage, which makes a therapeutic effect unpredictable.

Mosaicplasty involves the harvest of plugs of bone and cartilage from a non-load-bearing portion of a joint, and these plugs are then inserted into the site of injury in a mosaic pattern. Since the procedure requires a high degree of surgical precision, it is, consequently, osteochondral autograft (Mosaicplasty) is generally only available at specialist clinics. However, it is advantageous in that it can be used to treat injury sites that slightly larger than those treatable by marrow stimulation. The procedure is further advantageous in that an injury site is repaired by hyaline cartilage, and consequently a predictable therapeutic effect can generally be obtained. However, it is still disadvantageous in that it causes damages to the healthy cartilage tissue.

Autologous chondrocyte implantation (ACI) is in practical use in Europe and the United States. This procedure, which is actually a two-step procedure, involves the culture and reimplantation of a patient's own cells. During the first step of the procedure, the surgeon removes a biopsy sample of cartilage from a non-load-bearing portion of a joint, then cartilage cells (chondrocytes) are isolated from the sample and cultured over a two-week period before being returned to the surgeon. In the second step of the procedure, the surgeon implants the cultured cells at the site of injury and, if necessary, seals the defect with a biological membrane such as autogenic periosteum. By using this procedure it is possible to reduce a total amount of excised healthy cartilage tissue as compared with the mosaicplasty procedure.

However, ACI is considered to be disadvantageous in that it causes damage to healthy cartilage tissue. Furthermore, since removed cartilage cells (chondrocytes) have to be cultured in vitro, and primary chondrocytes do not proliferate well with human serum, only by around 10 fold, it is necessary to use materials derived from an animal other than human (such as fetal bovine serum) or to use artificial materials (such as collagen gel derived from bovine skin); otherwise, it is possible to treat only a relatively small defect, though the surgical procedure is both a little bit invasive and complex.

Mesenchymal stem cells (MSCs) show great promise as a potential source of cells for cell-based therapeutic strategies, because of their capacity for extensive self-renewal and multipotency (Pittenger et al., 1999, Science. 284:143-7). In addition to the fact that bone marrow is considered to be a well-accepted source of MSCs (Prockop, D. J., 1997, Science. 276:71-4), various studies have been reported that MSCs can be isolated from various adult mesenchymal tissues such as synovial membrane (De Bari, C. et al., 2001, Arthritis Rheum. 44:1928-42), periosteum (Fukumoto, T. et al., 2003, Osteoarthritis Cartilage. 11:55-64), adipose tissue (Zuk, P. A. et al., 2002, Mol Biol Cell. 13:4279-95), muscular tissue (Cao et al., 2003, Nat Cell Biol. 5:640-6) and so on.

We previously reported that pellet weight reflects production of cartilage matrix (Sekiya, I., et al., 2002, Proc Natl Acad Sci USA. 99:4397-402). These results indicated that bone marrow-derived MSCs retain chondrocyte differentiation potential to form cartilage tissue in vitro. Therefore, MSCs are considered to be attractive materials for cartilage regeneration from the viewpoints of using autologous cells, causing minimal damage and invasiveness into healthy cartilage tissue, and the possibility of obtaining a sufficient number of cells for cartilage regeneration.

In a large number of animal transplantation studies, MSCs expanded ex vivo were able to differentiate into cells of the residing tissue and repair tissue damaged by trauma or disease (Awad et al., 1999, Tissue Eng. 5:267-77; Li and Huard, 2002, Am J. Pathol. 161:895-907). Despite a diverse and growing body of information regarding MSCs and their use in cell-based strategies, the mechanisms that govern MSC self-renewal and multilineage differentiation are not well understood and remain an active area of investigation.

For full-thickness articular cartilage defects, transplantation of MSCs in collagen gel with periosteum covering has been attempted. Although some studies have reported successful results (Adachi et al., 2002, J. Rheumatol. 29:1920-30; Wakitani et al., 2002, Osteoarthritis Cartilage. 10:199-206), a number of questions such as whether the donor cells differentiated into chondrocytes or how donor cells contributed to chondrogenesis still exist, limiting clinical applications for cartilage injury.

A meniscus is a tissue consisting of fibrous cartilage and collagen and plays a role in spreading a load from a femur, in absorbing impact, and in providing stability and smooth movement of a knee joint. It is known in the art that a meniscus injury is resulted from meniscus tear caused by trauma such as sprain and bruise. There are some methods for treating a meniscus injury, each of which is depending on the extent of the meniscus injury. In the case of a small meniscus injury (such as a small meniscus tear of the marginal region), a surgeon usually selects conservative medical management of the injury. In the case of a large meniscus injury, a surgeon carries on meniscus suture or removal operation.

### SUMMARY OF THE INVENTION

An object of the present invention is to provide a method for treating defects of articular cartilage or meniscus of a patient using in vivo chondrogenesis of synovium-derived MSCs.

The inventors previously compared human MSCs derived from a variety of mesenchymal tissues including bone marrow and determined that synovium-derived MSCs had greater ex vivo expansion and chondrogenic ability than MSCs from other tissues (Sakaguchi et al., 2005, Arthritis Rheum. 52:2521-9). This indicates that synovium-derived MSCs may be an optimal cell source for cartilage regeneration.

Therefore, the present invention provides a method for treating a disease associated with defects of cartilage or meniscus. In the present invention, the method for treating a disease associated with defects of cartilage or meniscus comprises the following steps:

culturing ex vivo autologous synovium-derived mesenchymal stem cells (MSCs);

implanting the MSCs such that said cartilage defect site or meniscal defect site is covered by the MSCs; and

regenerating cartilage tissue at the cartilage defect site or meniscal defect site in situ by differentiating the MSCs into cartilage cells.

### BRIEF DESCRIPTION OF THE DRAWINGS

FIG. 1 shows morphology of primary synovium-derived mesenchymal stem cells (MSCs) from rabbit during their expansion.

FIG. 2 shows the isolation and characterization of synovium-derived and bone marrow-derived MSCs cultured with human autologous serum or with fetal bovine serum.

FIG. 3 shows the characteristics of differentiation of the synovium-derived MSCs at Passage 1.

FIG. 4 shows the chondrogeneic ability of rabbit synovium-derived MSCs in vivo.

FIG. 5 shows the scheme for minimally invasive technique for cartilage defect using synovium-derived MSCs.

FIG. 6 shows macroscopic observation of the site of cartilage defect 1 d, 4, 8, 12, and 24 weeks after synovium-derived MSC transplantation.

FIG. 7 shows histological analyses of the site of cartilage defect after synovium-derived MSC transplantation at 1 day.

FIG. 8 shows low magnified histological analyses of the site of cartilage defect after synovium-derived MSC transplantation at 4 weeks.

FIG. 9 shows high magnified histological analyses of the site of cartilage defect after synovium-derived MSC transplantation at 4 weeks.

FIG. 10 shows histological analyses of the site of cartilage defect after synovium-derived MSC transplantation at 24 weeks.

FIG. 11 shows histological scores for cartilage defect after synovium-derived MSC transplantation.

FIG. 12 shows MR images of the cartilage defect sites.

FIG. 13 shows a schematic view of the operative technique of a local adherent technique.

FIG. 14 shows effective accumulation of the injected luciferase/LacZ double positive synovial MSCs at the site of resected meniscus.

FIG. 15 shows that the injected luciferase/LacZ double positive synovial MSCs were not detected in other organs than the knee which was implanted with the synovial MSCs.

FIG. 16 shows that the transplanted MSCs directly differentiated into meniscal cartilage cells.

### DETAILED DESCRIPTION OF THE INVENTION

The present invention is described below in detail.

In this study, the inventors isolated MSCs from the synovium. After expansion ex vivo, the inventors transplanted 1,1′-dioctadecyl-3,3,3′,3′-tetramethylindocarbocyanine perchlorate (DiI)-labeled MSCs into a full-thickness articular cartilage defect. Intensive histological analyses demonstrated that transplanted MSCs altered over a time course according to local micro environments which were classified into: bone zone, border of cartilage and bone, center of cartilage, superficial zone, and the adjacent area of native cartilage. The articular cartilage defect was repaired by in situ chondrogenesis of MSCs without prior induction by differentiation medium. This system makes it possible to define in detail cellular events after transplantation of MSCs into cartilage, and advances the clinical application of MSCs for cartilage injury.

Articular cartilage consists of hyaline cartilage and meniscus consists of fibrous cartilage. The present inventors further confirmed that human articular cartilage could be regenerated by transplantation of human synovial mesenchymal stem cells and that rat meniscus could be regenerated by transplantation of rat synovial stem cells.

Therefore, in this study, the inventors transplanted luciferase-labeled MSCs into a meniscal defect site. Intensive histological analyses demonstrated that transplanted MSCs altered over a time course according to local micro environments and differentiated into a meniscal cartilage. The meniscal defect was repaired by in situ chondrogenesis of MSCs without prior induction by differentiation medium.

Therefore, the method of the present invention aims to provide a method for treating a disease associated with defects of cartilage or meniscus. Specifically, the method for treating a disease associated with defects of cartilage or meniscus provided in the present invention comprises at least the following steps:

a step of culturing ex vivo an autologous synovium-derived mesenchymal stem cells (MSCs);

a step of implanting the MSCs such that said cartilage defect site or meniscal defect site is covered by the MSCs; and

a step of regenerating cartilage tissue at the cartilage defect site or meniscal defect site in situ by differentiating the MSCs into the cartilage cells (chondrocytes). In the present invention, the transplanted MSCs differentiate into the chondrocytes according to local micro environments. As a result of the in situ chondrogenesis of MSCs, the cartilage tissue is regenerated at the cartilage defect site or meniscal defect site to repair the defect and, in the case of the cartilage defect, to form a bone zone, a border of cartilage and bone, a center of cartilage, a superficial zone, and an adjacent area of native cartilage as the native cartilage tissue or, in the case of the meniscal defect, to form meniscal cartilage.

In the present invention, a disease associated with defects of cartilage or meniscus to be treated by the method of the present invention is selected from the group consisting of, but not limited to, traumatic cartilage injury, osteochondritis dissecans, aseptic osteonecrosis, osteoarthritis, and meniscal injury.

In the context of the present invention, mesenchymal stem cells (MSCs) are known to reside in bone marrow, synovium, periosteum, adipose tissue, muscular tissue and to have an ability to differentiate into osteoblasts, chondrocytes, adipocytes, and myocytes. In relation to the differentiation of MSCs into chondrocytes, it is known that the differentiation of the undifferentiated MSCs into chondrocytes is facilitated by supplementation of BMP and TGF-β into the culture medium and, therefore, the cartilage tissue can be generated in an in vitro condition.

The transplanted cells used in the method of the present invention are undifferentiated MSCs. Our previous study demonstrated that synovium-derived MSCs exhibit the highest chondrogenic ability among various MSCs (including bone marrow-derived MSCs, periosteum-derived MSCs, adipose tissue-derived MSCs, muscular tissue-derived MSCs) (Sakaguchi et al., 2005, Arthritis Rheum. 52:2521-9). This indicates that synovium-derived MSCs may be an optimal cell source for in situ cartilage regeneration. Therefore, it is preferable to use the synovium-derived MSCs to be transplanted in the method of the present invention. Further, from the viewpoint of preventing the patient from generating the allograft rejection after implantation, it is preferable to use the autologous synovium-derived MSCs in the method of the invention.

It is known in the art that the MSCs may differentiate into cartilage cells to generate cartilage tissue in vitro when the MSCs are cultured in the chondrogenesis medium supplemented with transforming growth factor-β3 (TGF-β3), dexamethasone, and bone morphogenetic protein 2 (BMP-2). Therefore, in the present invention, it is preferable that, in order not to differentiate the MSCs into the chondrocytes, the isolated MSCs are cultured in the absence of TGF-β3, dexamethasone, or BMP2.

It is also known in the art that the synovium-derived MSCs decrease the in situ chondrogenic ability in reverse proportion to the passage number of the MSCs in vitro. Therefore, in order to prepare the cultured MSCs which are undifferentiated, it is preferable that the MSCs are used at Passage 0 or Passage 1.

Synovium tissue to be cultured ex vivo is harvested under anesthesia from a non-load-bearing portion of the joint. The excised synovium tissue is digested by protease(s) (such as collagenase and trypsin) and digested cells are filtered through a mesh filter (such as a 70-μm nylon filter). Nucleated cells isolated by the above method are used as synovium-derived MSCs in the present invention. In the case of using autologous serum, for example, the surgeon may obtain the patient's blood at the same time as obtaining the synovium tissue from the patient, or at another time.

The autologous synovium-derived MSCs isolated from the patient suffering from defects of cartilage or meniscus are cultured ex vivo without prior induction by differentiation medium (such as αMEM without supplementing TGF-β3, dexamethasone, or BMP2). The proliferated, undifferentiated synovium-derived MSCs are then transplanted back to the patient from whom the synovium-derived MSCs are derived. To efficiently cure the cartilage defect site or meniscal defect site using the proliferated MSCs, it is necessary to apply at least 5×10⁷ undifferentiated MSCs, more preferably, at least 1×10⁸ MSCs, per cartilage defect site or meniscal defect site about the area of 10 cm² for the efficient treatment of the cartilage defect site or meniscal defect site using MSCs.

With respect to the relations between the culture period and the chondrogenic ability of the cultured MSCs, it is known in the art that differentiation of the synovium-derived MSCs into cartilage cells proceeds as the culture period becomes longer and, therefore, in situ chondrogenic ability of the synovium-derived MSCs decreases if the culture period exceeds a certain length. Thus, in the present invention, in order to expand the synovium-derived MSCs being undifferentiated and having good in situ chondrogenic ability, it is preferable to adjust a culture period. Further, in the present invention, it is necessary to consider the necessity of preparing a certain number of undifferentiated MSCs sufficient to cover the cartilage defect site and to regenerate the affected site. Therefore, the isolated MSCs are cultured in vitro for 5-28 days, most preferably for 14-28 days, before the implantation. Further, in the present invention, it is necessary to culture the MSCs until tens of million of the cells are obtained.

The thus cultured undifferentiated MSCs are implanted at the cartilage defect site or meniscal defect site such that the cartilage defect site or meniscal defect site is covered by the MSCs. The implantation of the MSCs may be conducted by an open technique or by endoscopic operation. To limit invasiveness as far as possible, it is preferable to implant the MSCs endoscopically.

The cartilage defect site or meniscal defect site may be covered by the cell sheet of the MSCs, or by a suspension of the MSCs. For example, bioabsorbable gels, such as gelatin and collagens, may be used as the gel-like material. The MSCs are highly-adhesive to the cartilage defect site or meniscal defect site. Consequently, the present invention provides a novel, minimally invasive technique for treating a defect of cartilage or meniscus.

In the case of the treatment of the cartilage defect, the minimally invasive technique of the invention is characterized by covering said cartilage defect site by the MSCs comprising the following steps:

holding the body position to orient the cartilage defect site upward;

placing a cell sheet of the MSCs, a suspension of the MSCs or gel-like material containing the MSCs on the surface of an articular cartilage defect site; and

maintaining the body position for a certain period so that the MSCs adhere to the surface of the cartilage defect site.

In the case of the treatment of the meniscal defect, the minimally invasive technique of the invention is characterized by covering said meniscal defect site by the MSCs comprising the following steps:

holding the body position to orient the meniscal defect site downward;

injecting a suspension of the MSCs into the knee joint; and

maintaining the body position for a certain period to adhere the MSCs to the defect site.

To ensure adhesion of the MSCs to the surface of the cartilage defect site or meniscal defect site, it is desirable to hold the transplanted MSCs on the surface of the cartilage defect site or meniscal defect site for at least 10 minutes, and preferably for 15 minutes. To accomplish this, the body position is maintained for at least 10 minutes, and preferably for 15 minutes, in order to orient the cartilage defect site or meniscal defect site upward and to hold the MSCs at the upwardly oriented cartilage defect site or meniscal defect site.

The cartilage defect site or meniscal defect site with the MSCs may further be covered by periosteum in order to enhance adhesion of the MSCs to the cartilage defect site or meniscal defect site more assuredly. After holding the MSCs on the surface of the cartilage defect site or meniscal defect site for at least 10 minutes, the operation is brought to completion.

In the present invention, the transplanted MSCs differentiate into the chondrocytes at the cartilage defect site or meniscal defect site and regenerate in situ the cartilage tissue at the cartilage defect site or meniscal defect site. During the in situ chondrogenesis of MSCs, there is no necessity to take any external action since the cartilage tissue will regenerate in accordance with local micro environments (such as nutrient supply and cytokines' environments). As a result of the in situ chondrogenesis of MSCs, the cartilage tissue is regenerated at the cartilage defect site or meniscal defect site to repair the defect and, in the case of the cartilage defect, to form a bone zone, a border of cartilage and bone, a center of cartilage, a superficial zone, and the adjacent area of native cartilage as the native cartilage tissue or, in the case of the meniscal defect, to form meniscal cartilage.

As described above, the present inventors demonstrated that a disease associated with defects of cartilage or meniscus (such as traumatic cartilage injury, osteochondritis dissecans, aseptic osteonecrosis, osteoarthritis, and meniscal injury) can be treated using mesenchymal stem cells (MSCs). Therefore, in the present invention, a preparation for treating a disease associated with defects of cartilage or meniscus can also be provided. The preparation is characterized by comprising MSCs which are to be transplanted to the cartilage defect site or meniscal defect site.

The examples to be treated by the preparation above includes, but not limited to, traumatic cartilage injury, osteochondritis dissecans, aseptic osteonecrosis, osteoarthritis, and meniscal injury.

While the present invention has been described in relation to certain of its preferred embodiments, the following examples are provided by way of further illustration of the present invention but are by no means intended to limit its scope.

In the Examples, to assess differences, 2-way analysis of variance (ANOVA) and Students t test were used. A value of P<0.05 was considered significant

### EXAMPLES

### Example 1

### Isolation of Rabbit Synovium-Derived MSCs

This example is provided to describe a method for obtaining synovium-derived MSCs from rabbits.

Skeletally mature Japanese White Rabbits weighing about 3.2 kg (ranging 2.8-3.6 kg) were used in the experiments. Animal care was strictly in accordance with the guidelines of the animal committee of Tokyo Medical and Dental University. Synovium was harvested under anesthesia induced by intramuscular injection of 25 mg/kg of ketamine hydrochloride and intravenous injection of 45 mg/kg of sodium pentobarbital.

Harvested rabbit synovium was digested in a 3 mg/ml collagenase D solution (Roche Diagnostics, Mannheim, Germany) in αMEM (Invitrogen, Carlsbad, Calif., USA) at 37° C. After 3 hours digestion, digested cells were filtered through a 70-μm nylon filter (Becton Dickinson, Franklin Lakes, N.J., USA) and the remaining tissues were discarded.

The obtained cells were plated at 5×10⁴ cells/cm² in 60-cm² culture dishes (Nalge Nunc International, Rochester, N.Y., USA) in complete culture medium: αMEM supplemented with 10% FBS (Invitrogen; lot selected for rapid growth of bone marrow-derived mesenchymal stem cells (MSCs)), 100 units/ml penicillin (Invitrogen), 100 μg/ml streptomycin (Invitrogen), and 250 ng/ml amphotericin B (Invitrogen) and incubated in cell incubator at 37° C. with 5% CO₂ in a humidified atmosphere. The medium was changed every 3-4 days to remove non-adherent cells and then cultured for 14 days as Passage 0 without refeeding. Then the cells were trypsinized, harvested and replated as Passage 1 at 50 cells/cm² in 145-cm² culture dishes (Sekiya, I., et al., 2002, Stem Cells. 20:530-41). After an additional 14 days of growth, the harvested cells were resuspended at a concentration of 1×10⁶ cells/ml in αMEM with 5% dimethylsulfoxide (Wako, Osaka, Japan) and 20% FBS to cryopreserve. Aliquots (1 ml) were slowly frozen and cryopreserved in liquid nitrogen (Passage 1). To expand the cells, a frozen vial of the cells was thawed, plated in a 145-cm² culture dish with the complete culture medium, and incubated 4 days in the recovery plate at 37° C. with 5% CO₂ in humidified atmosphere. These cells (Passage 2) were used for further analyses.

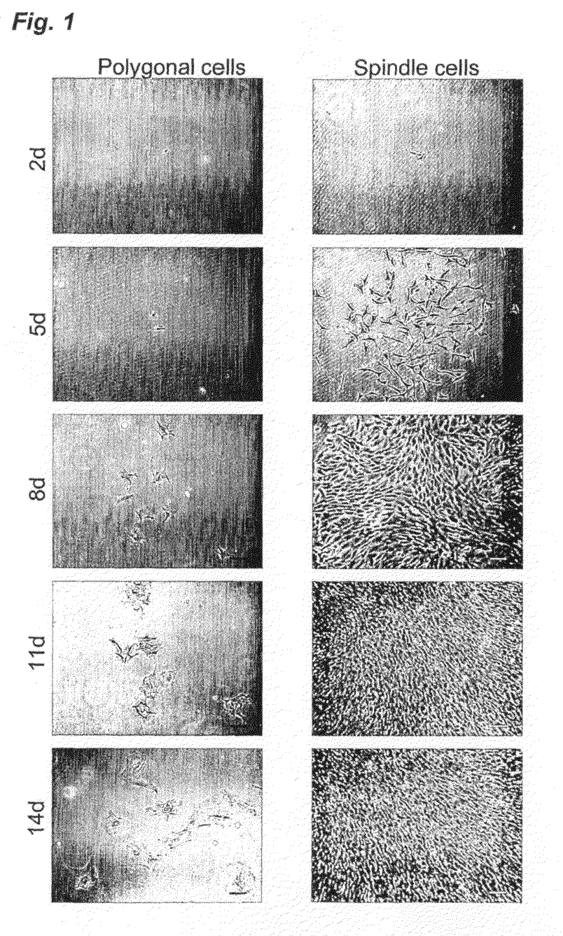

Sequential observation of the adherent cells demonstrated two types of single cell-derived colonies, polygonal cells and spindle cells; larger dense colonies consisting of small spindle cells and smaller dim colonies comprised of larger polygonal cells (FIG. 1). Cells were photographed on the days noted (Bar: 100 μm). The spindle cells proliferated much more rapidly than the polygonal cells; consequently, the population comprised a majority of spindle cells after 14 days.

### Example 2

### Isolation and Characterization of Human Synovium-Derived MSCs with Autologous Human Serum

In this example, the inventors isolated human synovium-derived MSCs and bone marrow-derived MSCs and identified the characterization thereof.

### (i) Isolation of Human MSCs and Proliferative Effect Thereof

The study was approved by the local institutional review board, and all human study subjects provided informed consent. Human synovium and bone marrow were harvested during the operation of anterior cruciate ligament (ACL) reconstruction of the knee from 8 donors (27±5 years old).

Bone marrow from the tibia was obtained with an 18-gauge needle just before drilling for the insertion of reconstructed ligaments. Synovium with subsynovial tissue from the inner side of the medial joint capsule, which overlies the noncartilaginous areas of the medial condyles of the femur, was harvested with a pituitary rongeur, under arthroscopic observation. One day before the operation of ACL reconstruction, 100 ml of total blood were harvested from all donors, and human serum was separated. Nucleated cells from the bone marrow were isolated with a density gradient (Ficoll-Paque; Amersham Biosciences).

Synovium were digested in a 3 mg/ml collagenase D solution (Roche Diagnostics) in Hank's balanced salt solution (HBSS; Invitrogen) at 37° C. After 3 hours, digested cells were filtered through a 70-μm nylon filter (Beckton Dickinson) and the remaining tissues were discarded.

Nucleated cells from synovium were plated at 10⁴ cells/cm² and those from bone marrow were plated into a 10 cm dish as a clonal density cultured in a complete culture medium: alpha-modified Eagle's medium (α-MEM; Invitrogen), 100 units/ml penicillin, 100 μg/ml streptomycin, and 250 ng/ml amphotericin B (all from Invitrogen) containing 10% autologous human serum or 20% fetal bovine serum (lot selected for rapid growth of bone marrow-derived MSCs). Briefly, we prepared 4 groups of cells at primary culture: 1) synovial MSCs cultured with autologous human serum, 2) synovial MSCs cultured with FBS, 3) bone marrow MSCs cultured with autologous human serum and 4) bone marrow MSCs cultured with FBS. Cells of 4 groups were harvested 14 days after plating with 0.25% trypsin and 1 mM EDTA (ethylenediaminetetraacetic acid; Invitrogen) for 5 minutes at 37° C., counted with a hemocytometer to determine the number of the cells at passage 0.

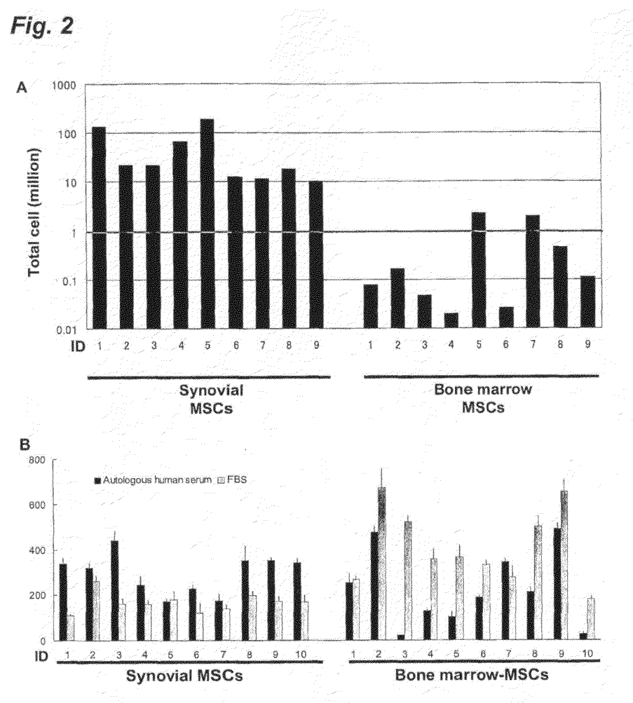

Total yields of primary human synovial and bone marrow MSCs cultured with autologous human serum were shown in FIG. 2A. Nucleated cells from 221±113 mg synovium or from 2±2 ml bone marrow were plated, cultured for 14 days, and harvested. These tissues were collected from 10 donors and total yields were shown respectively.

To determine the proliferative potential, cells from each of the 4 groups described above were plated at 50 cells/cm² as Passage 1 and cultured for 14 days with 10% autologous human serum or 20% FBS. 14 days after plating, the cells were harvested and counted.

Comparison of proliferative effect of human serum with fetal bovine serum on synovial and bone marrow MSCs at passage 1 is shown in FIG. 2B. Synovial MSCs and bone marrow MSCs from 10 donors were replated at 50 cells/cm², cultured for 14 days with autologous human serum or FBS, and fold increase with standard deviation is shown (n=3).

FIG. 2 shows that the human synovium-derived MSCs proliferate better in the presence of autologous human serum than in the presence of FBS. On the other hand, the bone marrow-derived MSCs proliferate better in the presence of FBS than in the presence of autologous serum. Of course, it is true that the bone marrow-derived MSCs can proliferate in the presence of autologous serum; however, the growth rate of the bone marrow-derived MSCs varies dramatically from cell to cell. Reviewing these data and considering that it is undesirable to use materials derived from an animal other than human, it is apparent that it is desirable to use the synovium-derived MSCs which are cultured in the presence of autologous serum as cells for regenerative therapy.

### (ii) Differentiation Assay

A MSC is defined as cells being derived from mesenchymal tissue and by its functional capacity to both self-renew, which is commonly identified by colony forming unit-fibroblast assay (Friedenstein, A. J., 1976, Int Rev Cytol. 47:327-59) and by its ability to generate a number of differentiated progeny (McKay, R., 1997, Science. 276:66-71; Prockop, D. J., 1997, Science. 276:71-4).

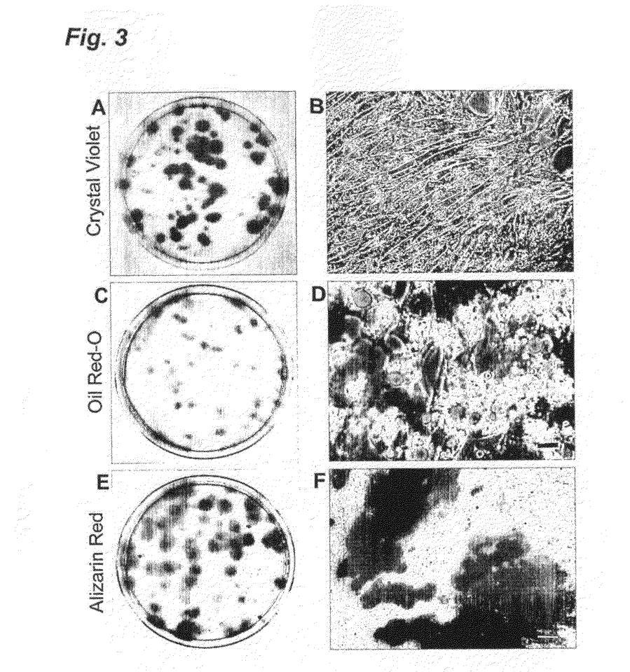

To examine the ability of cell colony formation, the synovium-derived cells at passage 1 were plated at 100 cells per 60-cm² culture dish in 6 dishes and cultured for 14 days to form cell colonies. Three dishes were stained for 5 minutes with 0.5% Crystal Violet in methanol. The cells were washed twice with distilled water and the number of colonies per dish was counted to assess the colony-forming efficiency (FIG. 3A). Colonies less than 2 mm in diameter and faintly stained colonies were ignored. The total number of cells was counted from 3 other dishes and the cell number per colony was calculated to assess the proliferation activity (Sakaguchi et al., 2004, Blood. 104:2728-35).

Larger dense colonies consisted of spindle cells as observed in FIG. 3B (Bar: 50 μm). The colony forming unit efficiency of the cells at Passage 1 was 60±5% (mean±SD, n=3) and the cell number per colony was 6774±437 cells.

For adipogenesis, 100 cells were plated in 60 cm² dishes and cultured for 14 days in αMEM-based complete medium to make cell colonies (as described above). The medium was then switched to an adipogenic medium that consisted of the complete medium supplemented with 10⁻⁷M dexamethasone (Sigma-Aldrich Corp. St. Louis, Mo., USA), 0.5 mM isobutylmethylxanthine (Sigma-Aldrich Corp.) and 50 μM indomethacin (Wako, Tokyo, Japan) and the cells were cultured for an additional 21 days. The adipogenic cultures were fixed in 4% paraformaldehyde, stained with fresh Oil Red-O solution, and the number of Oil Red-O-positive colonies was counted. Colonies less than 2 mm in diameter or faint colonies were ignored. The adipogenic cultures were subsequently stained with Crystal Violet, and the number of total cell colonies was counted (Sekiya, I, et al., 2004, J Bone Miner Res. 19:256-64). Adipocyte colonies were shown in red (FIG. 3C) and higher magnification of Oil Red-O-positive cells is also shown in FIG. 3D (Bar: 25 μm).

For osteogenesis, 100 cells were plated in 150 cm² dishes and cultured for 14 days in the complete medium. The medium was then switched to a calcification medium that consisted of the complete medium supplemented with 10⁻⁹ M dexamethasone, 20 mM β-glycerol phosphate (Wako), and 50 μg/ml ascorbate-2-phosphate (Sigma-Aldrich Corp.) and the cells were cultured for an additional 21 days. These dishes were stained with 0.5% Alizarin Red solution, and the number of alizarin red-positive colonies was counted. The calcification cultures were subsequently stained with Crystal Violet, and the number of total cell colonies was counted. Colonies less than 2 mm in diameter or yellowish colonies were ignored (Sakaguchi et al., 2004, Blood. 104:2728-35). Calcified colonies were shown in red (FIG. 3E); higher magnification of Alizarin Red-positive cells is also shown in FIG. 3F (Bar: 250 μm).

Oil Red-O positive colony rate for adipogenesis was 74±6% (n=3) and Alizarin Red positive colony rate for osteogenesis was 79±6% (n=3).

### Example 3

### Chondrogenic Ability of Synovium-Derived MSCs

This example is provided to identify the chondrogenic ability of rabbit synovium-derived MSCs ex vivo.

For ex vivo chondrogenesis, 250,000 cells were placed in a 15-ml polypropylene tube (Becton Dickinson, Franklin Lakes, N.J., USA) and were centrifuged at 450 g for 10 min. The pellet was cultured in a chondrogenesis medium consisting of high-glucose Dulbecco's modified Eagle's medium (DMEM high glucose; Invitrogen Corp, Carlsbad, Calif., USA) supplemented with 500 ng/ml BMP-2 (bone morphogenetic protein-2; Yamanouchi Pharmaceutical, Tokyo, Japan), 10 ng/ml TGF-β3 (transforming growth factor-β3; R&D Systems. Minneapolis, Minn., USA), 10⁻⁷M dexamethasone (Sigma-Aldrich Corp. St. Louis, Mo., USA), 50 μg/ml ascorbate-2-phosphate, 40 μg/ml proline, 100 μg/ml pyruvate, and 1:100 diluted ITS+Premix (BD Biosciences. Bedford, Mass., USA; 6.25 μg/ml insulin, 6.25 μg/ml transferrin, 6.25 ng/ml selenious acid, 1.25 mg/ml BSA, and 5.35 mg/ml linoleic acid). For microscopy, the pellets were embedded in paraffin, cut into 5-μm sections, and stained with Toluidine Blue.

Passage 2 cells were resuspended at 1×10⁶ cells/ml in αMEM and the fluorescent lipophilic tracer 1,1′-dioctadecyl-3,3,3′,3′-tetramethylindocarbocyanine perchlorate (DiI; Molecular Probes, Eugene, Oreg., USA) was added at 5 μl/ml in αMEM. After incubation for 20 minutes at 37° C. with 5% humidified CO₂, an aliquot of the cells was centrifuged at 450 g for 10 min. The pellet was cultured in the chondrogenesis medium. For fluorescent microscopy, the pellets were embedded in paraffin, cut into 5-μm sections. Nuclei were counterstained by DAPI (Sekiya, I., et al., 2001, Biochem Biophys Res Commun. 284:411-8; Sekiya, I., et al., 2005, Cell Tissue Res. 320:269-76).

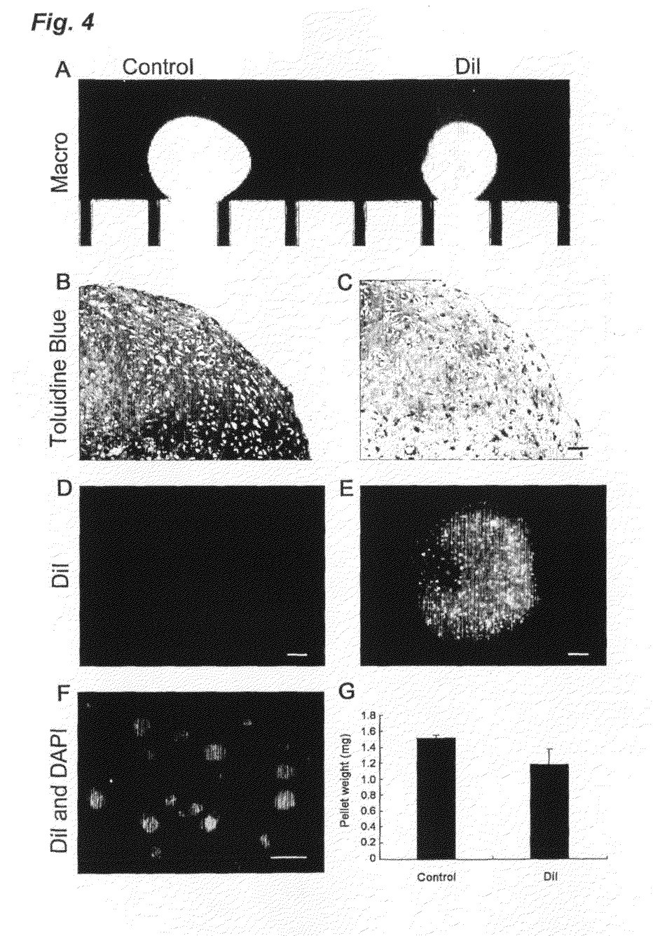

Macro picture of pellets are shown with a 1 mm scale (FIG. 4A). The cell pellet increased its size over the culture period and had a spherical, transparent appearance at 21 days (FIG. 4A, left). DiI-labeled cell pellets also grew and became spherical, but were pinkish under gross observation (FIG. 4A, right).

Histological section of pellet of unlabeled (FIG. 4B) and DiI-labeled cells (FIG. 4C) stained with Toluidine Blue. Cartilage matrix was examined histologically (FIGS. 4B and 4C). Histological analysis demonstrated the existence of cartilage matrix (FIG. 4B).

Fluorescent microscopic analysis of unlabeled (FIG. 4D) and labeled cells (FIG. 4E, FIG. 4F). The nuclei were counterstained by DAPI (FIG. 4F). Fluorescence was highly maintained without leakage of DiI to extracellular matrix for at least 21 days (FIGS. 4E, 4F).

The wet weight of pellets of unlabeled and labeled cells (FIG. 4G). The weights of pellets from DiI-labeled cells were lighter than those derived from control cells (FIG. 4G).

We previously reported that pellet weight reflects the production of cartilage matrix (Sekiya, I., et al., 2002, Proc Natl Acad Sci USA. 99:4397-402). These results indicated that rabbit synovium-derived MSCs retain chondrocyte differentiation potential after the labeling of DiI, but that it suppressed chondrogenesis in some degree.

Bars indicate 50 μm (FIGS. 4B, 4C); 250 μm (FIGS. 4D, 4E); 25 μm (FIG. 4F). The data is expressed as mean±SD. p<0.05 by unpaired t-test (n=3).

### Example 4

### Development of a Novel, Minimally Invasive Technique for Treating Cartilage Defect

In this example, a novel, minimally invasive technique for treating cartilage defect is provided.

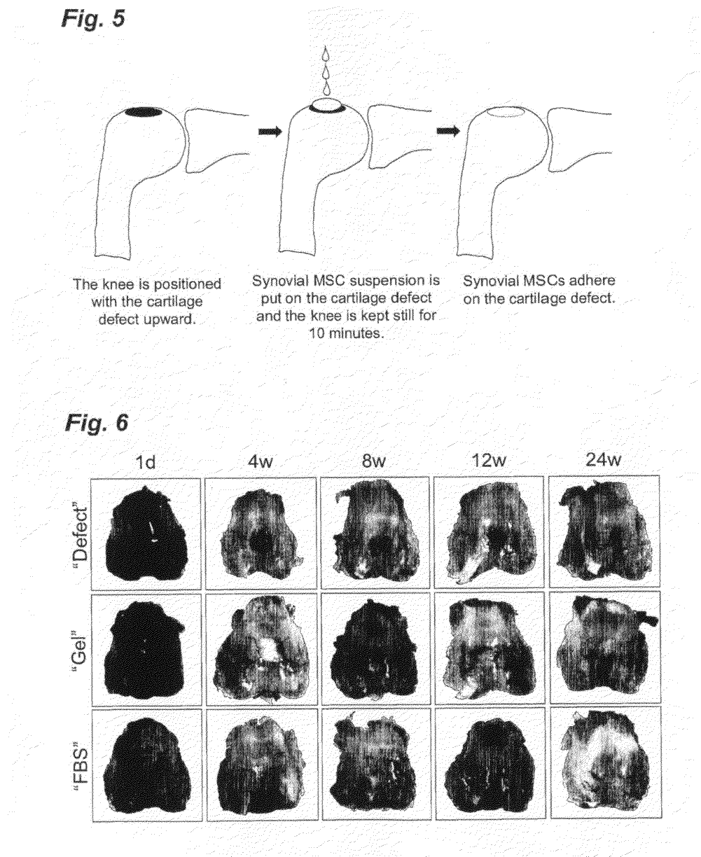

The scheme of the novel, minimally invasive technique for treating cartilage defect is shown in FIG. 5.

The MSCs are highly-adhesive to the cartilage defect site. To hold the MSCs at the cartilage defect site, the body position may be held to orient the cartilage defect site upward, and the MSCs may then be positioned at the upwardly oriented cartilage defect site.

Then, the cartilage defect site was covered by a suspension of the MSCs or by the MSCs embedded in collagen gel. After holding the MSCs on the surface of the cartilage defect site for 10 minutes, the operation was brought to completion.

In the present example, the cartilage defect site with the MSCs was further covered by periosteum in order to enhance adhesion of the MSCs to the cartilage defect site.

### Example 5

### In Vivo Transplantation and Histological Examination

In this example, the inventors conducted macroscopic observation of cartilage defect after transplantation of synovium-derived MSCs.

Skeletally matured Japanese White Rabbits weighing about 2.9 kg (ranging 2.6-3.3 kg) were used in the experiments. Animal care was in strict accordance with the guidelines of the animal committee of Tokyo Medical and Dental University.

Synovium and total blood were harvested under anesthesia induced by intramuscular injection of 25 mg/kg of ketamine hydrochloride and intravenous injection of 45 mg/kg of sodium pentobarbital. Rabbit serum was isolated from total blood by using the same methods as human serum.

Synovium tissue was harvested from left knee and digested in a 3 mg/ml collagenase D solution in HBSS at 37° C. After 3 hours, digested cells were filtered through a 70-μm nylon filter and the remaining tissues were discarded. Nucleated cells were plated in 3 dishes (150 mm diameter) and cultured in β-MEM supplemented with antibiotics and 10% autologous rabbit serum or 20% fetal bovine serum. Fourteen days after plating the cells, the cells were harvested as MSCs with 0.25% trypsin and 1 mM EDTA for 5 minutes at 37° C. and counted with a hemocytometer to determine the number of the cells.

The cells were DiI labelled in accordance with the method described in Example 3. DiI was also used to detect transplanted cells in animal study as described in the following. The collected DiI-labeled cells were centrifuged at 450 g for 5 min, washed twice with PBS, and 5×10⁶ DiI-labeled cells were re-suspended in 50 μl of αMEM with 20% FBS. They were then mixed with an equal volume of collagen gel (Atelocollagen, 3% type 1 collagen, Koken, Tokyo, Japan) and embedded in 100 μl of collagen gel-MSCs mixture at the concentration of 5×10⁷ cells/ml, which are used for transplantation.

Operation was performed under anesthesia. The detailed procedure of the technique for implanting the MSCs on the cartilage defect site is described in Example 4 above. The rabbits were anesthetized again by intramuscular injection of 25 mg/kg of ketamine hydrochloride and intravenous injection of 45 mg/kg of sodium pentobarbital, the right knee joint was approached through medial prapatellar incision, and the patella was dislocated laterally. Full thickness osteochondral defects (5×5 mm wide, 3 mm deep) were created in the trochlear groove of the femur and the animals were divided into 4 groups: “Defect”, “Gel”, “FBS”, and “Autologous serum” groups.

In the “Defect” group, the defect sites were not covered by any materials. In the “Gel” group, the defect sites were filled with a mixture containing an equal volume of αMEM with 20% FBS and collagen gel, which does not contain any cells in it. In the “FBS” group, the defect sites were filled with DiI-labeled autologous MSCs dispersed and embedded in collagen gel at the concentration of 5×10⁷ cells/ml, which are cultured in α-MEM supplemented with 20% FBS. In the “autologous serum” group, the defect sites were filled with DU-labeled autologous MSCs dispersed and embedded in collagen gel at the concentration of 5×10⁷ cells/ml, which are cultured in αMEM supplemented with 10% autologous serum. In the “FBS” and “Autologous serum” groups, the defect sites were further covered with periosteum. All rabbits were returned to their cages after the operation and were allowed to move freely and to eat and drink ad libitum.

Animals were sacrificed with an overdose of sodium pentobarbital at 1 day, 4, 8, 12, and 24 weeks after the operation. Samples were first examined macroscopically regarding color, integrity and smoothness. Osteoarthritic changes and synovitis of the knee were also investigated. After examination, the distal femurs were dissected and were then photographed. Femoral condyles at 1 day and 4, 8, 12, and 24 weeks after the surgery are shown in FIG. 6.

At 1 day, the cartilage defect was overlaid with blood clots in the “Defect” group; while, in the “FBS” and “Autologous serum” groups, the cartilage defect site was covered by a layer of the MSCs. At 4 weeks, the center of the defect site appeared as slightly whitish in the “Defect” group; while, in the “FBS” and “Autologous serum” groups, the cartilage defect site was filled with the cartilage tissue derived from the transplanted MSCs. In the “FBS” and “Autologous serum” groups, continuity between the regenerated cartilage tissue and the neighboring cartilage tissue appeared better than that of the “Defect” group.

In the “Defect” group, the cartilage defect had a patchy whitish appearance at 8 weeks, looked smaller at 12 weeks, and even smaller at 24 weeks. However, the defect was still observed. In the “Gel” group, the border between periosteum and the neighboring cartilage became smoother after 8 weeks. However, periosteum was still observed distinctly at 24 weeks. Mild osteophyte formation was observed on the edge of the trochlear groove in some samples of the “Defect” and “Gel” groups. In the “FES” and “Autologous serum” groups, cartilage defect covered with periosteum appeared glossy, smooth, and similar with neighboring cartilage at 8 weeks and the margin of the repaired tissue appeared to be integrated into the surrounding native cartilage at 12 weeks and thereafter (FIG. 6).

### Example 6

### Histological Examination and Fluorescent Microscopic Examination

In this example, the inventors conducted histological examination and fluorescent microscopic examination.

The dissected distal femurs were fixed in a 4% paraformaldehyde solution immediately. The specimen was decalcified in 4% EDTA solution, dehydrated with a gradient ethanol series, and embedded in paraffin blocks. Sagittal sections (5 μm thick) were obtained from the center of each defect, and stained with toluidine blue. Sections dedicated for fluorescent microscopic visualization of DiI labeled cells were not stained with toluidine blue, and nuclei were counterstained with DAPI.

Immunohistochemical examination was conducted as follows. Paraffin-embedded sections were deparaffinized using xylene and dehydrated through graded alcohols. The samples were pretreated with 0.4 mg/ml proteinase K (DAKO, Carpinteria, Calif., USA) in Tris-HCl for 15 minutes at room temperature for antigen retrieval. Residual enzymatic activity was removed by washing in PBS and non-specific staining was blocked with PBS containing 10% normal horse serum for 20 minutes at room temperature. Primary antibodies (type 1 and type 2 collagen; Daiichi Fine Chemical, Toyama, Japan) were placed on the sections for one hour at room temperature. After extensive washing with PBS, biotinylated horse anti-mouse IgG (Vector Laboratories, Burlingame, Calif., USA) was placed as a secondary antibody on the sections for 30 minutes at room temperature. Immunostaining was detected with VECTASTAIN ABC reagent (Vector Laboratories), followed by DAB staining. Counterstaining was performed with Mayer-hematoxylin.

In this Example, the inventors obtained histological data at 1 day, 4, 8, 12, and 24 weeks after surgery. As representative results, histological data at 1 day, 4 and 24 weeks after implantation are shown.

(i) Histological Analyses at 1 Day after Implantation:

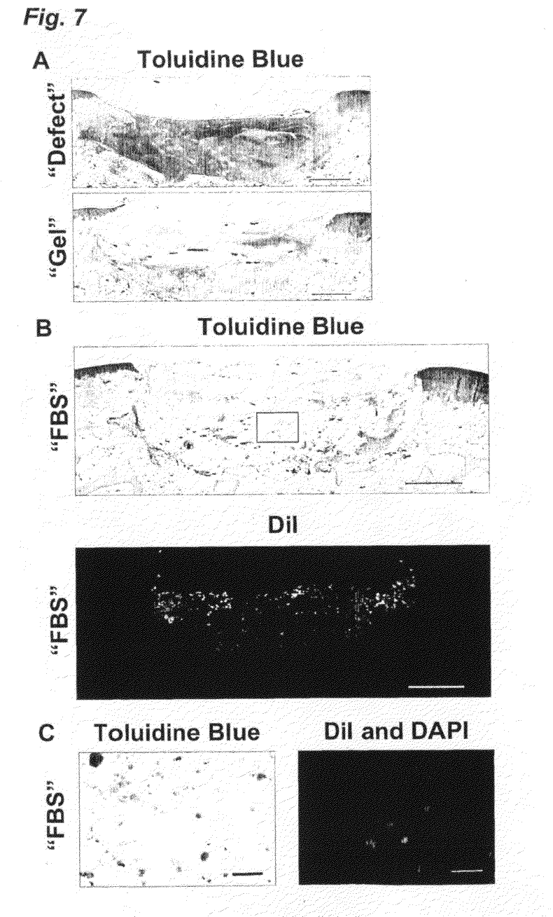

Histological analyses at 1 day after implantation are shown in FIG. 7. Sagittal sections of the cartilage defect stained with Toluidine Blue in “Defect” group (FIG. 7A, upper), “Gel” group (FIG. 7A, lower) and the MSCs of “FBS” group (FIG. 7B, upper) at 1 day. Serial section of “FBS” group under epifluorescent microscopy is shown as FIG. 7B, lower.

At 1 day, in the “Defect” group, the defect was filled with blood clot (FIG. 7A, upper). In the “Gel” group, collagen gel was covered with periosteum in the defect and blood clot filled in between gel and trabecular bone (FIG. 7A, lower). In the “FBS” group, the defect was filled with collagen gel containing MSCs and covered with periosteum (FIG. 7B, upper). DiI labeling and nuclear staining with DAPI confirmed the presence of transplanted MSCs in the defect area in the “FBS” group (FIG. 7B, lower).

Higher magnifications of the framed area in FIG. 7B upper panel stained with Toluidine Blue (FIG. 7C, left) and under epifluorescent microscopy regarding the “FBS” group (FIG. 7C, right). The nuclei were counterstained by DAPI in Fig. C, right panel. Distal side was located at right side. Bars indicate 1 mm (FIGS. 7A and 7B); 50 μm (FIG. 7C).

(ii) Histological Analyses at 4 Weeks after Implantation:

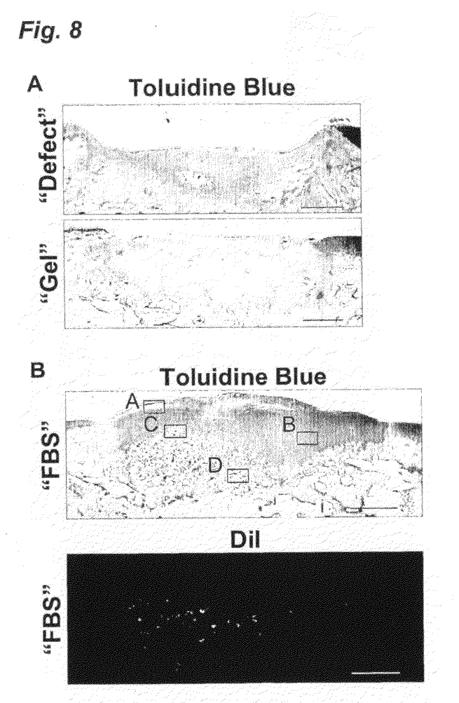

Sagittal sections of the defects stained with Toluidine Blue in “Defect” group (FIG. 8A, upper) and “Gel” group (FIG. 8A, lower) at 4 weeks. At 4 weeks, in the “Defect” group, fibrous tissue partially filled in the defect (FIG. 8A, upper). In the “Gel” group, periosteum still remained (FIG. 8A, lower) and collagen gel with a few cells was observed (data not shown). Chondrocyte-like cells were partially detected at the peripheral area of the defect (data not shown), however, their cartilage matrix formation appeared poor.

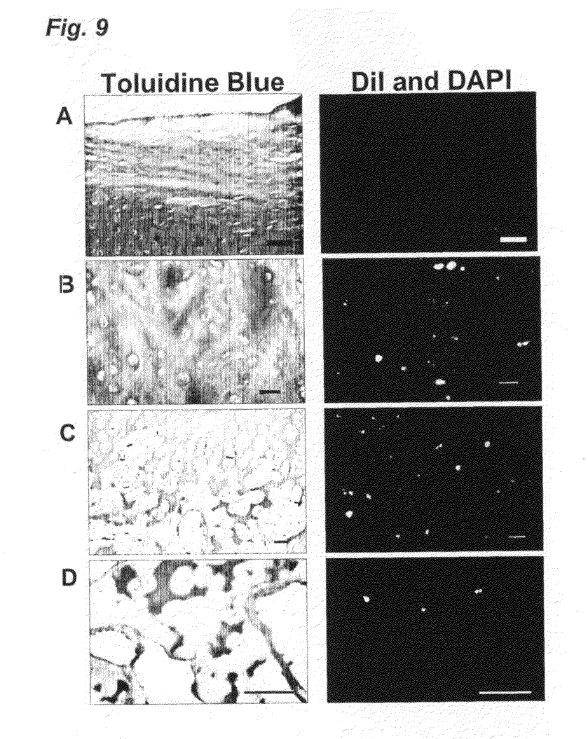

Higher magnification of the framed area in FIG. 8B, upper panel, stained with Toluidine Blue (FIG. 9, left) and under epifluorescent microscopy (FIG. 9, right) are also shown. The nuclei were counterstained with DAPI (FIG. 9, right). The distal side is located at the right side. Bars indicate 1 mm (FIG. 8); 50 μm (FIGS. 9B and 9D); 25 μm (FIGS. 9A and 9C).

In the “FBS” group, most of the defect and the periosteum were filled with cartilage matrix (FIG. 9B, upper). Though the number of DiI positive cells decreased (FIG. 9B, lower), they differentiated into chondrocytes (FIG. 9A). The remnant of periosteum became thinner and the amount of the cartilage matrix at the remnant of periosteum (FIG. 9B) was less than that at the center of regenerated cartilage (FIG. 9A). Cells at the remnant of periosteum were DiI negative, however, a number of chondrocytes adjacent to the remnant of periosteum were DiI positive (FIG. 9B). DiI positive hypertrophic chondrocytes were observed at the deep area of the cartilage zone (FIG. 9C). The deep area of the defect was partially replaced with newly formed trabecular bone and some cells composing bone were also DiI positive (FIG. 9D); to the contrary, cells in the medullary cavity were DiI negative.

(iii) Histological Analyses at 24 Weeks after Implantation:

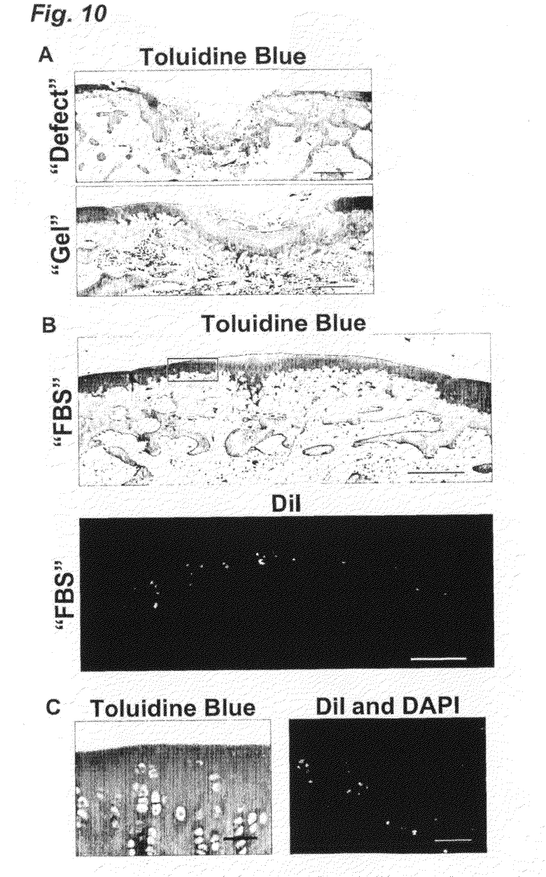

Sagittal sections of the defects stained with Toluidine Blue in “Defect” group (FIG. 10A, upper), “Gel” group (FIG. 10A, lower), and “FBS” group (FIG. 10B, upper) at 24 weeks. Serial section of “FBS” group was observed under epifluorescent microscopy (FIG. 10B, lower). Higher magnifications of the framed area in FIG. 10B, upper, are shown in FIG. 10C, in which the section stained with Toluidine Blue is shown in FIG. 10C, left, and the section observed under epifluorescent microscopy is shown in FIG. 10C, right. The nuclei were counterstained with DAPI in FIG. 10C, right. The distal side is located at the right side. Bars indicate 1 mm (FIGS. 10A and 10B); 50 μm (FIG. 10C).

At 24 weeks, in the “Defect” and “Gel” groups, the cartilage was poorly healed (FIG. 10A). In the “FBS” group, the subchondral bone was remodeled to form an osteochondral junction and the thickness of the regenerated cartilage was almost the same as that of native cartilage (FIG. 10B). Integration between native and regenerated cartilage was improved and could not be distinguished at the proximal side. DiI-positive cells still remained at the cartilage zone (FIG. 10B, right, and 10C).

### Example 7

### Histological Score of Cartilage Regeneration

In this example, the inventors examined histological score of cartilage regeneration.

Blinded histological observations were quantified using a histological grading scale for the defect of cartilage described previously (Wakitani et al., 1994, J Bone Joint Surg Am. 76:579-92) (Table 1).

TABLE I
Histological grading scale for the defects of cartilage^{a}

| Category | Points |
|---|---|
| Cell morphology | |
| Hyaline cartilage | 0 |
| Mostly hyaline cartilage | 1 |
| Mostly fibrocartilage | 2 |
| Mostly non-cartilage | 3 |
| Non-cartilage only | 4 |
| Matrix-staining (metachromasia) | |
| Normal (compared with host adjacent cartilage) | 0 |
| Slightly reduces | 1 |
| Markedly reduced | 2 |
| No metachromatic stain | 3 |
| Surface regularity^{b} | |
| Smooth (>¾) | 0 |
| Moderate (½-¾) | 1 |
| Irregular (¼-½) | 2 |
| Severely irregular (<¼) | 3 |
| Thickness of cartilage^{c} | |
| >⅔ | 0 |
| ⅓-⅔ | 1 |
| <⅓ | 2 |
| Integration of donor with host adjacent cartilage | |
| Both edge integrated | 0 |
| One edge integrated | 1 |
| Neither edge integrated | 2 |
| Total maximum | 14 |

^{a}Described by Wakitani et al., 1994.
^{b}Total smooth area of the reparative cartilage compared with the entire area of the cartilage defect.
^{c}Average thickness of the reparative cartilage compared with that of the surrounding cartilage.

The histological score was determined by the blinded histological observations. A full score was 14 and a lower score indicated improvement. The data are expressed as mean±SD (n=3) (Table 2).

TABLE 2
Histological Score

| | 4 w | 8 w | 12 w | 24 w |
|---|---|---|---|---|
| “Defect” group | 11.7 | 9.5 | 9.5 | 8.0 |
| “Gel” group | 11.5 | 7.7 | 11.0 | 10.3 |
| “FBS” group | 4.5 | 2.3 | 1.3 | 1.3 |

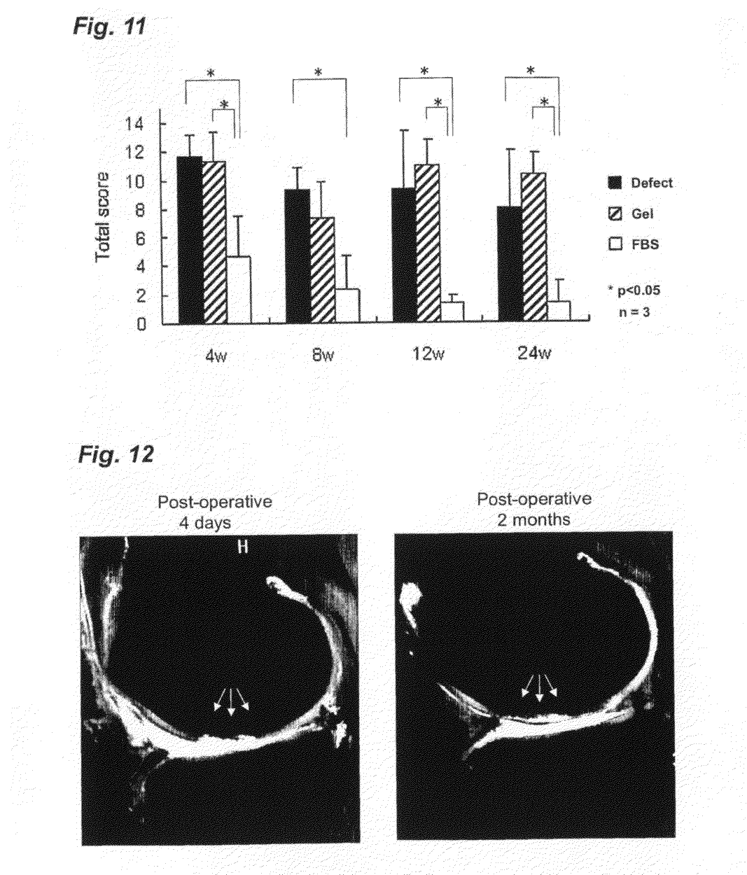

The scores of the “FBS” groups in each time point were significantly higher than the “Gel” group and the “Defect” group except for the “Gel” group at 8 weeks and “Defect” group at 24 weeks (FIG. 11).

### Example 8

### Novel Implantation Procedure with Synovial MSCs for Cartilage Regeneration in Human

For discoveries of novel drugs and medical procedures, even if animal studies provide promising data, the clinical studies often demonstrate disappointing outcome or unexpected adverse effects. This means that the outcome of an animal study does not correspond to the outcome of clinical study. This is due to the differences of function of cells and tissues between in human and other animals. Therefore, even if certain hypothesis is true in an animal experiment, we definitely need to confirm in human for clinical application.

Therefore, in this example, another novel, minimally invasive technique for treating cartilage defect is provided. The study was approved by the local institutional review board, and all human study subjects provided informed consent.

The patient was 25 years male with cartilage defect of medial femoral condyle. One day before the harvest of synovium, 100 ml of whole blood was drawn from a patient in the closed bag system (JMS Co., Ltd, Hiroshima, Japan). The system consists of a blood donation bag containing glass beads. The glass beads in a bag function as platelet activator as well as removal of fibrin from whole blood through a gently mixing process for 30 minutes. After centrifuging at 2,000 G for 7 minutes, the serum was isolated, heat-inactivated at 56° C. for 30 minutes, and stored at −20° C.

Synovium with subsynovial tissue from the inner side of the medial joint capsule was harvested from the same patient with a pituitary rongeur arthroscopically under spinal anesthesia.

Thus obtained synovium (0.2 g) was digested in a solution containing 3 mg/ml collagenase in Hank's balanced salt solution (HBSS; Invitrogen, Carlsbad, Calif.) at 37° C. After 3 hours, the digested cells were filtered through a 70-μm nylon filter (Beckton Dickinson). Nucleated cells (13 million) were plated on 25 dishes of 150-cm² and cultured in the complete culture medium: alpha-modified Eagle's medium (α-MEM; Invitrogen), 100 units/ml penicillin, 100 μg/ml streptomycin, and 250 ng/ml amphotericin B (Invitrogen) containing 10% autologous human serum for 14 days. The cells were harvested with TrypLE (Invitrogen) at 37° C. for 15 minutes, counted with a hemocytometer to determine the number of the cells at passage 0.

Autologous synovial MSCs were transplanted 14 days after the harvest. Under spinal anesthesia, fibrous tissue on the cartilage defect was removed arthroscopically. The cartilage defect of the femoral condyle was faced upward. Irrigation fluid was totally evacuated. The defect was filled with the suspension of the autologous synovial MSCs, which consisted of 40 million cells in 1 ml lactate ringer (Lactec, Otsuka Pharmaceutical Co., Tokyo, Japan), by slow injecting with 1 ml syringe. The knee was held to keep the position for 10 minutes.

One day after the operation, range of motion of the knee and partial weight-bearing exercises were begun. The patient walked without crutches at 4 weeks after the operation. He had undergone MRI examinations at 4 days and 2 months after the operation.

MR imaging at 4 days showed cartilage defect of medial femoral condyle, while MR imaging at 2 months demonstrated that the cartilage defect was filled with cartilaginous tissue (FIG. 12).

### Example 9

### Meniscal Regeneration by Exogenous Synovial MSCs in Rat Massive Meniscectomy Model

In this example, the inventors examined meniscal regeneration by implanting synovial MSCs. All experiments were conducted in accordance with the institutional guidelines for the care and use of experimental animals.

Male luciferase/LacZ double transgenic rats were anesthetized by an intra-peritoneal injection of sodium pentobarbital (25 mg/kg) and synovial tissues were harvested from knee joints. The tissues were minced, digested with type V collagenase (0.2%; Sigma, Lakewood, N.J.) for 3 hours at 37° C., and passed through a 70-μm filter (Becton Dickinson, Franklin Lakes, N.J.). Nucleated cells from synovium were plated at 10⁴ cells/150 cm²-dish and cultured in complete medium (αMEM, Invitrogen, Carlsbad, Calif.; 20% FBS, lot-selected for rapid growth of human MSCs, Invitrogen; 100 units/ml penicillin, 100 μg/ml streptomycin, 250 ng/ml amphotericin B, and 2 mM L-glutamine, Invitrogen) for 14 days. Then, the cells were harvested after treatment with 0.25% trypsin and 0.02% EDTA, counted using a hemocytometer, and replated at 50 cells/cm². The cells were collected after 14 days and frozen at −80° C. with Cryo 1° C. Freezing Container (Nalgene Nunc International, Rochester, N.Y.) at 10⁶ cells in 1 ml solution as Passage 1. The stocked cells were thawed in a water bath at 37° C. rapidly, plated in 150 cm²-dish, and harvested 3 to 4×10⁶ cells after 5 days. Then, passage 2 cells were replated at 10⁴ cells/cm², cultured for 14 days, and collected for further analyses and transplantation at Passage 3.

Rats (Sprague-Dawley rats; n=30) were used and anesthetized. A straight skin incision was made on the anterior side of the knee, the anteromedial side of the joint capsule with the medial collateral ligament was cut horizontally at the level of the knee joint, and the anterior horn of the medial meniscus was resected.

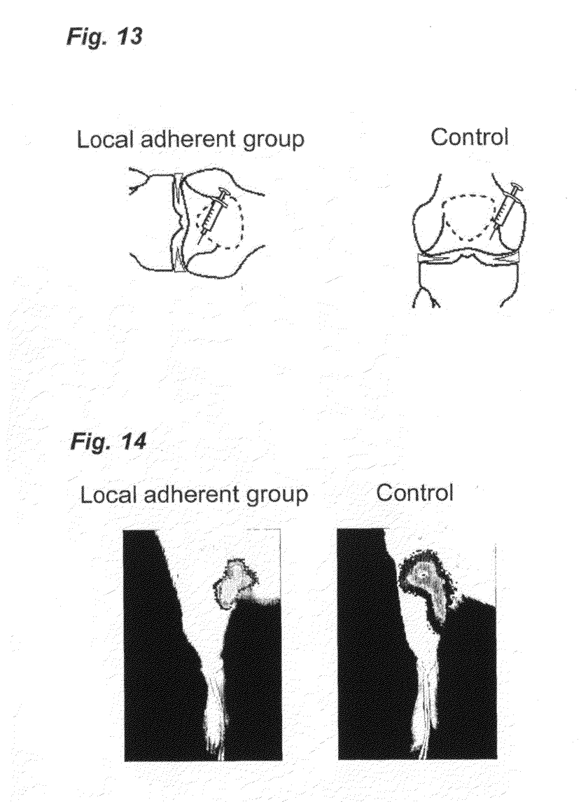

Just after the skin incision was closed, a 27-gauge needle was inserted at the center of the triangle formed by the medial side of patellar ligament, the medial femoral condyle, and the medial tibial condyle, toward the intercondylar space of the femur. Then, 5×10⁶ luciferase/LacZ double positive synovial MSCs in 50 μl PBS were injected into the right knee joint. For control, the same volume of PBS was injected into the left knee joint and flexed and extended 5 times and was laid down in the supine position for 10 minutes (FIG. 13).

For local adherent group, the knee was positioned with the resected meniscus downward (decubitus position) and held to keep the position for 10 minutes (FIG. 13). The rats were allowed to walk freely in the cage.

The injected luciferase/LacZ double positive synovial MSCs were detected by IVIS imaging system and X-gal staining. The regenerated meniscus was evaluated macroscopically.

IVIS imaging system conducted one day after the injection demonstrated that the injected luciferase/LacZ double positive synovial MSCs accumulated to the site of resected meniscus more effectively with local adherent technique than with intraarticular injection (FIG. 14).

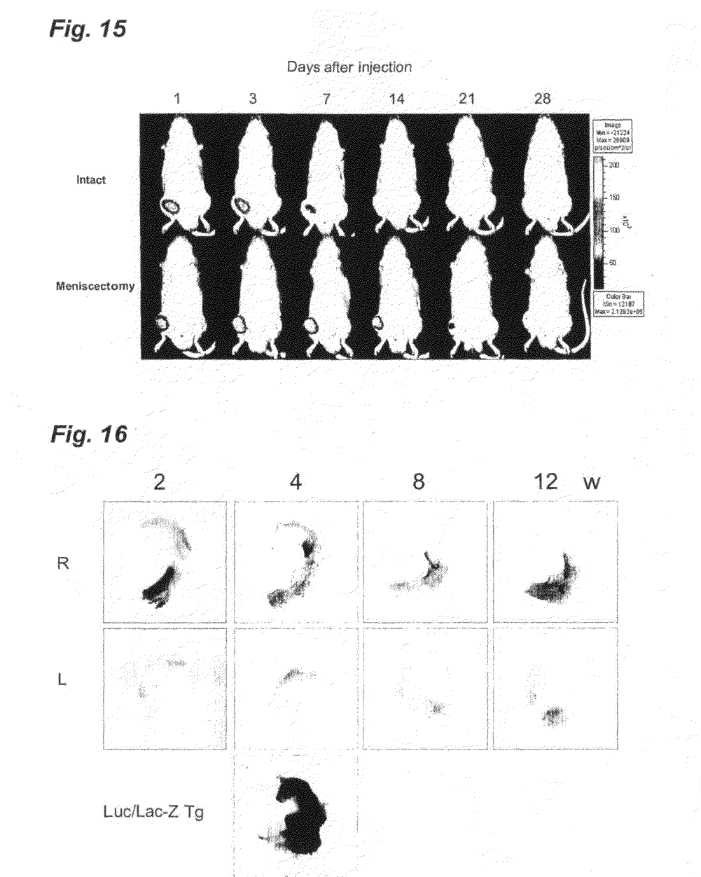

The cells injected into menisectomized knee were detected for a longer period than the cells injected into intact knee. Importantly, the injected luciferase/LacZ double positive synovial MSCs were not detected in other organs except the right knee (FIG. 15).

The injected synovial MSCs enhanced meniscal regeneration, which were LacZ positive, demonstrating transplanted MSCs directly differentiated into meniscal cells (FIG. 16).

Articular cartilage consists of hyaline cartilage and meniscus consists of fibrous cartilage. We confirmed that human articular cartilage could be regenerated by transplantation of human synovial stem cells and that rat meniscus could be regenerated by transplantation of rat synovial stem cells. These naturally make researchers in this field think that transplantation of human synovial stem cells promotes regeneration of human meniscus defect.

1. A method for treating a disease associated with defects of cartilage or meniscus, wherein said method comprises:
culturing ex vivo an autologous synovium-derived mesenchymal stem cells (MSCs);
implanting the MSCs such that said cartilage defect site or meniscal defect site is covered by the MSCs; and
regenerating cartilage tissue at the cartilage defect site or meniscal defect site in situ by differentiating the MSCs into the cartilage cells (chondrocytes).

2. The method according to claim 1, wherein said disease associated with defects of cartilage or meniscus is selected from the group consisting of traumatic cartilage injury, osteochondritis dissecans, aseptic osteonecrosis, osteoarthritis, and meniscal injury.

3. The method according to claim 1, wherein the MSCs are cultured in the absence of bone morphogenetic protein (BMP) or transforming growth factor-β (TGF-β).

4. The method according to claim 1, wherein the MSCs are cultured for 5-28 days before the implantation.

5. The method according to claim 1, wherein the MSCs are cultured until tens of million of the cells are obtained.

6. The method according to claim 1, wherein the MSCs are used at Passage 0 or Passage 1.

7. The method according to claim 1, wherein the step of covering said cartilage defect site by the MSCs is conducted by the steps of:
holding the body position to orient the cartilage defect site upward;
placing a suspension of the MSCs on the surface of an articular cartilage defect site; and
maintaining the body position for a certain period to adhere the MSCs to the surface of the cartilage defect site.

8. The method according to claim 1, wherein the step of covering said meniscal defect site by the MSCs is conducted by the steps of:
holding the body position to orient the meniscal defect site downward;
injecting a suspension of the MSCs into the knee joint; and
maintaining the body position for a certain period to adhere the MSCs to the defect site.

9. The method according to claim 1, wherein the suspension of the MSCs or the gel-like material containing the MSCs is placed on an articular cartilage defect site for at least 10 minutes.

10. A preparation for treating a disease associated with defect of cartilage or meniscus, which comprises human MSCs.

11. The preparation according to claim 10, wherein said disease associated with defect of cartilage or meniscus is selected from the group consisting of traumatic cartilage injury, osteochondritis dissecans, aseptic osteonecrosis, osteoarthritis, and meniscal injury.
